(19) United States
(12) Patent Application Publication
(10) Pub. No.: US 2026/0306712 A1
(43) Pub. Date: Oct. 8, 2026

(54) THERMOGELLING POLYMERS ABLE TO FORM A SLIPPERY COATING FOR AN OPHTHALMIC INJECTOR

(71) Applicant: POLYMEREXPERT, Pessac (FR)

(72) Inventors: Marc DOLATKHANI, Cestas (FR); Anne PAGNOUX, Le Barp (FR)

(21) Appl. No.: 19/481,572

(22) PCT Filed: May 3, 2024
(86) PCT No.: PCT/FR2024/050584

§ 371 Date: Nov. 5, 2025

(30) Foreign Application Priority Data
May 5, 2023 (FR) FR2304533

(51) Int. Cl. A61L 27/34; A61L 27/50

(52) U.S. Cl. CPC A61L 27/34; A61L 27/50; A61L 2420/02

(57) ABSTRACT

The application relates to the use of heat-sensitive polymers capable of forming thermoreversible gels in order to form a slippery coating on at least one part of an intraocular implant injection device, in particular the tip and/or the cartridge, allowing injection via micro-incision.

### TECHNICAL FIELD

The invention relates to the preparation of a slippery coating intended to coat at least one part of an intraocular implant injection device, in particular the tip and/or the cartridge, allowing injection via micro-incision. The main property of this coating is that of allowing the folded implant to slide along the tip and/or the cartridge, while ensuring that it is not detached from its substrate and carried into the eye.

### PRIOR ART

The lens of a cataract-affected eye is replaced by intraocular implants. Surgery using phacoemulsification makes it possible to destroy the natural lens and remove it via a small incision. Implants made of soft, foldable materials capable of being inserted using injection devices via the micro-incision made for phacoemulsification have been developed.

The injection system is composed of a tubular body wherein the injection plunger slides, topped with a conical-shaped tip, the diameter of which decreases on moving closer to the injection end (tip).

The surgeon presses on the plunger, the end of which pushes the implant; the latter is increasingly constrained in the injector tip and eventually exits the injector completely folded. This makes it possible to inject an implant of more than 6 mm in diameter via an incision of less than 3 mm. This places very high stress on the implant during transfer. To limit the injection force and allow the implant to exit the tip without damage, it is necessary to optimize the tip geometry and the nature of the substrate, on the one hand, and to use a “lubricant”, on the other hand.

In the remainder of the description, the terms “intraocular implant injection device”, “injection system” or “ophthalmic injector” will be used interchangeably.

Patent applications EP1173115, EP2344073 or WO/2007 054644 and WO/2007 021412, for example, describe injection systems with different geometric designs. Many commercially available injectors relate to the different mechanical forms and/or inventions described in the patents. Mention may be made, for example, of one-piece injectors (connected tips and injection bodies) such as Skyjet® sold by Carl Zeiss Meditec, or Accujet®, Navijet® or Viscojet® injectors from Medicel AG where the tip is removable and consists of an injection chamber wherein the implant is placed, which is immediately folded and passes via a cannula to be injected, or Monarch@type injectors from Alcon Laboratories where the tip is removable and the implant is slid into the tip without prior folding.

The mechanical characteristics of the constituent material of the tip also have an influence on injectability. Indeed, the thermoplastic material used for manufacturing the tip must allow a certain deformability to accompany the stress exerted on the implant while having a good rigidity. In addition, the thermoplastic materials used must be able to be injected at high speeds. These materials are preferably chosen in the family of polypropylenes, polyamides, polyurethanes or polyesters and more particularly in the family of polypropylenes and polyamides.

However, the choice of material of the tip and/or the cartridge and optimizing its geometry are not sufficient to inject implants via micro-incisions satisfactorily. It is essential to use a lubricant allowing the implant to slide into the tip and/or the cartridge. Alternatively, the tip and the cartridge may be integral and form a single element. This alternative is covered by the expression “the tip and/or the cartridge” or “tip/cartridge”.

Several different approaches allowing sliding are described in the literature.

The first relates to the use of a blooming agent integrated by compounding into the thermoplastic material. It consists of a glycerol monostearate (GMS) type low molar mass organic surfactant molecule which is compounded with polypropylene or polyamide. It is distributed uniformly in the thermoplastic substrate just after injection and eventually migrates to the surface of the substrate after several days or even several weeks. This blooming phenomenon is linked with the small size of the surfactant molecule which is mobile compared to macromolecular chains. By way of example, U.S. Pat. No. 6,733,507 describes polypropylene cartridges containing a lubricating agent that migrates to the surface by a blooming phenomenon.

This approach has two major drawbacks.

The first is the presence of white marks on the injected implants. They are due to the blooming agent which is not bound to the tip surface and which is carried during injection. Indeed, the blooming agents used are not water-soluble and their removal is only possible after numerous rinses once the implant has been injected.

The second drawback is linked with the blooming phenomenon of the blooming agent on the substrate surface. This blooming may take several days or even several weeks, depending on the conditions of use (injection of the part), storage temperature, post-treatment, sterilization conditions, etc., before a sufficient quantity of lubricant is found on the surface of the injection cartridge. Lubrication quality will therefore depend on the wait time between the manufacture of the injector and its use by the practitioner. If this period is too short, lubrication is not provided satisfactorily, and if it is too long, the implant is covered with white spots (presence of blooming agent) during injection. To overcome this drawback, application WO2005/018505 proposes to heat-treat parts in order to accelerate the blooming phenomenon and to obtain a quasi-stable state (sufficient quantity of lubricant on the substrate surface) allowing reproducible injections. Another way described by patent U.S. Pat. No. 7,348,038 suggests a plasma treatment to provide a physical bond of the blooming agent to the cartridge.

Non-reproducibility of lubrication and the presence of whitish spots on implants during injection are the main failings of the use of blooming agents.

To avoid these drawbacks, application WO2013/083935 describes a metastable polymer composition comprising a mixture of at least one constituent polymer and at least one functional copolymer which are partially miscible or compatible, wherein
said constituent polymer is a thermoplastic polymer having a glass transition temperature (Tg) or a melting temperature (Tf) greater than or equal to 80° C., and is present in the mixture in a proportion by mass between 85% and 99.5%, and
said functional copolymer has a glass transition temperature (Tg) or a melting temperature (Tf) greater than or equal to 40° C., has at least 60% by mass of hydrophilic monomer units, and hydrophobic sequences or blocks partially miscible or compatible with the constituent polymer. This composition makes it possible to manufacture a biomedical device having a slippery surface without requiring coating of a material. The principle is based on blooming of the functional copolymer within the constituent polymer, which requires the use of a low molar mass functional copolymer, capable of migrating to the surface of the constituent polymer.

In application FR2986532, it is mentioned that the purpose sought is totally different from the “coating” approach (p. 16, 1.8-15):

“The present invention is totally different from the “coating” approach.

The latter, which consists in depositing a polymer film on the surface of the device or some of its elements, requires that several operations be carried out including surface activation, deposition of the polymer in solution and solvent evaporation. The approach proposed in the present invention makes it possible to avoid all these steps in the manufacturing process and results in a simplification of the method to the sole injection implementation step.”

Thus, in application FR2986532, blooming of the metastable polymer is quasi-immediate after the tip implementation operation, which ensures reproducibility of the slippery properties. In addition, the strong interactions between the metastable polymer and the constituent thermoplastic polymer of the tip allow very good adhesion and prevent the metastable polymer from being carried into the eye at the time of implant injection.

Another approach allowing implants to slide into injector tips/cartridges is to place a hydrophilic coating inside the tip. The lubrication principle consists in swelling the hydrophilic coating by adding a viscous product (hyaluronate or hydroxypropylmethyl cellulose solution) and thus sliding over a film of water formed at the interface.

Patent applications or patents JP5690838, JP3254752, U.S. Pat. No. 5,716,364, EP1949871, WO96/22062, WO2007/030009, WO2010/118080, U.S. Pat. Nos. 7,687,097, 7,348,038, WO2010/059655, for example, describe the possibility of producing a coating making it possible to reduce or remove friction between the implant and the cartridge.

The hydrophilic coating is bound either by covalent chemical bonds or by physical bonds to the surface of the tip and/or cartridge.

In relation to coatings bound by physical interactions to the substrate, surface activation by plasma or corona or other photochemical methods is systematically recommended as described, for example, in patent applications JP5690838, JP3254752, EP1949871, U.S. Pat. No. 5,716,364, WO2010/059655, WO2010/118080, or U.S. Pat. No. 7,348,038. Once the surface is activated, the polymer in solution is deposited and the solvent is then evaporated.

The hydrophilic polymers are selected from polyacrylic acid, polymethacrylic acid, polyvinylacetate, polyacrylamide, polyvinylpyrolidone, and their copolymer, and sometimes a mixture of several of these polymers.

However, the constituent polymers of the coating may detach from the substrate or be solubilized by the viscous product and be carried by the implant into the eye. If the practitioner does not rinse the eye satisfactorily after implant placement, residues from the coating may eventually cause inflammation.

Coatings covalently bound to the cartridge and/or the tip are moreover described, particularly in patent applications or patents U.S. Pat. No. 7,687,097, and WO96/22062. The recommended approach consists in immersing the cartridge optionally pre-treated with plasma in a precursor having reactive functions, for example acrylate groups, then by a radical pathway (thermal or UV irradiation) initiating the polymerization of the precursor of which some chains react with the radicals formed on the cartridge surface.

Other patent applications or patents such as U.S. Pat. Nos. 6,238,799, 6,866,936, or WO96/23602 describe covalent grafting of hydrophilic polymers, or hydrogels, on medical device substrates. The principle is based on the development of interpenetrated networks consisting of a polyurethane associated with another hydrophilic polymer.

However, covalent grafting of coating to the surface of the ophthalmic injector tip and/or cartridge is a lengthy and costly operation which limits the interest of the approach.

It is therefore sought to develop a slippery coating which does not require chemical grafting to the surface of the tip and/or the cartridge of the injector and which does not detach during implant injection.

Application WO2022/112730 describes the use of a polymer capable of forming a slippery coating for an ophthalmic injector. High molar masses combined with an adjusted hydrophilic-hydrophobic balance of the constituent polymer of the coating give it good slippery properties without detaching and without being carried into the eye during implant injection.

Application EP1521795 describes heat-sensitive polymers capable of forming thermoreversible gels in water. These polymers are formed from linear chains of polyoxyethylene-polyoxypropylene-polyoxyethylene triblock copolymer with organic groups bound at one or both ends via carbamate and urea bonds. Application EP1521795 also relates to their use in therapeutic or non-therapeutic compositions, in particular cosmetics. However, their use as a coating (in film form) of a medical device is not described in this application.

### DISCLOSURE OF THE INVENTION

The invention relates to the use of heat-sensitive polymers capable of forming thermoreversible gels in order to form a slippery coating on at least one part of an intraocular implant injection device.

In particular, the invention relates to the use of polymers formed of linear chains of polyoxyethylene-polyoxypropylene-polyoxyethylene triblock copolymer with organic groups bound at one or both ends via carbamate and/or urea bonds, as described in application EP1521795 as a coating of the inner surface of one or more parts of an intraocular implant injection device, making it possible to make it slippery, as well as the method for coating said surface, allowing good polymer adhesion. The invention also relates to a part of an intraocular implant injection device, the inner surface of which is coated with a polymer as defined above, as well as an intraocular implant injection device comprising at least one part of which the inner surface is coated with said polymer.

### BRIEF DESCRIPTION OF THE FIGURES

FIG. 1 shows the rheological flow (10 s⁻¹) characterization between 10° C. and 50° C. of the polymer of Example 1 in solution in water at a concentration of 7.5%.

FIG. 2 shows the rheological flow (10 s⁻¹) characterization between 10° C. and 50° C. of the polymer of Example 2 in solution in water at a concentration of 7.5%.

### DETAILED DESCRIPTION

The aim of the present invention is to provide a slippery coating for intraocular implant injection devices, said coating being prepared from LCST (Lower Critical Solubility Temperature) thermogelling polymers

They consist of water-soluble heat-sensitive polymers capable of forming thermoreversible physical gels with a high viscosity index, which comprise both chain formations comprising at least one heat-sensitive polyoxyalkylene triblock type linear chain consisting of polyethylene oxide (PEO) blocks and polypropylene oxide (PPO) blocks, said chain having the form PEO-PPO-PEO and being extended at at least one of its ends by an organic group via a carbamate bond and/or a urea bond.

The invention therefore relates to the use of the water-soluble heat-sensitive polymers described above in order to form a slippery coating on at least one part of an ocular implant injection device, in particular on the inner surface of said part.

“Thermoreversible physical gel” means a polymer lattice of which inter-chain bonds are provided by non-covalent bonds, in this case by Hydrogen bonds.

“High viscosity index polymer” means a polymer, which in solution (in this case, in solution in water) allows the solution to reach high viscosities i.e. more than 10 Pa·s under 10 s⁻¹ shear for a polymer concentration of 7.5%.

Said polymer has, in solution in water at a concentration less than 10%, a low viscosity at ambient temperature and is capable of forming physical gels with a high viscosity index at a temperature greater than 25° C. (thermogelling).

Said polymer is formed of heat-sensitive hydrophobic parts and hydrophilic parts. The formation of the gel is explained by the self-association of heat-sensitive portions in hydrophobic micro-domains, the entire polymer being maintained in solution by the hydrophilic segments.

Said polymers are sufficiently hydrophilic to be swollen by the viscous products used during eye surgery (sodium hyaluronate gel, hydroxy propyl methyl cellulose) while being sufficiently hydrophobic to avoid any solubilization at body temperature. Indeed, thermogelling from 25° C. creates strong physical interactions between the polymer chains resulting in the formation of a non-water-soluble physical gel. This physical cross-linking prevents any unintentional dispersion of said coating into the patient's eye, thus preventing post-operative problems.

Thus, according to one of its aspects, the invention relates to the preparation of an ophthalmic injector coating from a polymer wherein the hydrophilic-hydrophobic balance and the thermogelling capacity from 25° C., resulting in a physical gel, are perfectly adapted in order to form a film on the surface of an ophthalmic injector tip.

This film also has the specificity of being slippery thanks to the hydrophilicity of the polymer, on the one hand, and of being perfectly water-resistant thanks to its thermogelling property (formation of a non-water-soluble physical lattice), on the other hand. This water-resistance prevents any entrainment of the polymer into the eye and ensures good adherence to the ophthalmic injector which is in contact with the eye and the temperature of which at the time of injection into the eye is greater than 25° C.

Unlike application WO2022/112730, wherein the polymer must have a sufficiently high molar mass (number average molar mass between 20,000 g/mol and 1,000,000 g/mol and preferably between 50,000 g/mol and 500,000 g/mol), in order to prevent its solubilization in water during its use, the polymer described above may have much lower molar masses on the condition that it is thermogelling. This thermogelling must be based on the LCST of the polymer in order to allow the formation of a physical lattice. The formation of this physical gel prevents solubilization of the coating in aqueous medium during implant injection. The “slippery” property of the coating according to the invention can be measured, for example, using a one-piece ophthalmic injector entirely manufactured by injection/molding from said composition or an injector consisting of two parts wherein only the loading cartridge and the tip are manufactured from said composition. The thrust force on the injector plunger required to eject the implant loaded in the injector cartridge, via a tip wherein the outlet diameter is less than 3 mm, for example 2 mm, is measured. This measurement can be carried out by compression using an Instron 3367 type dynamometer equipped with a force sensor of 0.5 kN sensitivity at a speed of 4 mm/s and at ambient temperature. The slippery property is considered to be substantial for force values between 5 and 10 N. It is moderate for force values greater than 10 N and less than 15 N, and low for force values greater than 15 N.

Thus, the deposition of a coating according to the invention is considered to give an ophthalmic injector a slippery property when a practitioner injects, without damage, an implant with a diopter less than or equal to 30D into a patient's eye, by applying a force less than or equal to 20 N.

The invention also relates to the method for implementing the coating. The method according to the invention allows good adhesion of said polymer to the surface of the tip and/or the cartridge of the injector in order to prevent said coating from being detached and carried away by the implant, during its injection, into the patient's eye.

According to the invention, said coating is deposited on the surface of the tip and/or the cartridge of the injector. Its adhesion is essentially ensured by the hydrophobic blocks (PPO) of said polymer which have very good chemical compatibility with the constituent material of the tip and/or the cartridge of the injector.

The hydrophilic blocks (PEO) of said polymer, which have little or no interactions with the surface of the tip and/or the cartridge of the injector, make it possible to obtain the desired slippery properties.

One of the aims of this invention is to prepare a polymer having hydrophobic blocks having sufficiently strong interactions with the surface of the tip and/or the cartridge of the injector (without any interpenetration between polymer chains) and hydrophilic blocks making it possible to obtain sufficient slippery properties for intraocular implant injection. Advantageously, the slippery coating according to the invention has the following properties:
an adjusted hydrophilic-hydrophobic balance,
a thermogelling property from 25° C. resulting in its insolubility in water,
film-forming properties,
a good affinity with the substrate, for example made of polypropylene or polyamide.

According to one aspect of the invention, the constituent polymer of the coating according to the invention may be chosen from the family of thermogelling polyurethanes, particularly those described in application EP1521795. The adjustment of the hydrophilic-hydrophobic balance of this type of polymer can be obtained by the choice of type of PEO-PPO-PEO triblock polymer and diisocyanates.

The invention therefore relates, according to one of its aspects, to the use of a polymer as defined above consisting of a plurality of PEO-PPO-PEO triblock polyoxyalkylene type linear chains connected to each other by carbamate and/or urea bonds obtained from reacting the hydroxyl ends of PEO-PPO-PEO triblock polyoxyalkylene with a diisocyanate.

The chains of said polymer may be terminated by adding ethanol or extended by adding a polyethylene glycol monomethyl ether.

The polymer according to the invention preferably comprises a majority hydrophilic portion, preferably at least 70% by mass of PEO type hydrophilic monomer units.

Polyurethanes also have good film-forming properties; moreover, they are used as varnishes for many applications. Finally, their polarity and their capacity to create hydrogen bonds allow them to have a high affinity with many substrates.

The invention therefore relates, according to one of its aspects, to the use of a polymer consisting of a plurality of PEO-PPO-PEO triblock polyoxyalkylene type linear chains, of formula (I):

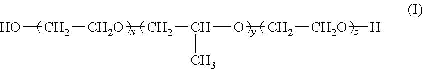

wherein, 10<x<150, 10<y<150, 10<z<150, said chains being connected to each other by carbamate and/or urea bonds obtained from reacting the hydroxyl ends of PEO-PPO-PEO triblock polyoxyalkylene with a diisocyanate, in order to form a slippery coating on at least one part of an ocular implant injection device, in particular on the inner surface of said part.

Preferably, x is equal to z. Preferably 10<x=z<150 and 15<y<60. The diisocyanates that can to be used for the synthesis of said polymers may particularly be chosen from the following compounds:

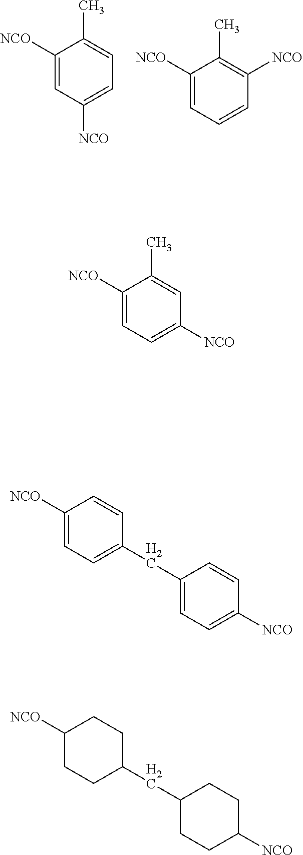

A preferred diisocyanate is, for example, dicyclohexylmethane-4,4′-diisocyanate.

The thermogelling polymers capable of forming a slippery coating for an ophthalmic injector may be prepared as described in application EP1521795.

In brief, the method comprises reacting at least one heat-sensitive polyoxyalkylene triblock type linear polymer of formula (I) having at least one terminal hydroxy function with at least one organic molecule carrying at least one isocyanate function so as to bind them together by carbamate or urea bonds.

Preferably, said obtaining method comprises reacting PEO-PPO-PEO triblock polyoxyalkylene with a diisocyanate, such that the polymer has at least 70% by mass of PEO.

The polyurethane chains of the polymer of the formula thus obtained may be either terminated by adding ethanol, or extended by adding a polyethylene glycol monomethyl ether comprising between 2 and 70 ethylene oxide units, so as to form the polymers of formula (II) below:

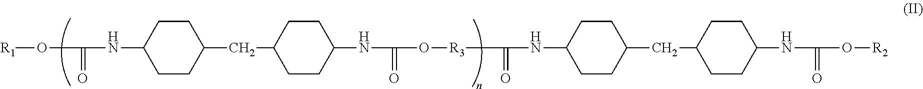

wherein:
   R₁ and R₂, identical or different, represent —(CH₂—CH₂—O)ₚ—CH₃ or —CH₂—CH₃;
   n is an integer from 2 to 50 and p is an integer from 2 to 70;
   R₃ represents formula (III)

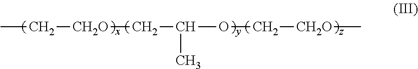

wherein x, y, and z are as defined above for formula (I).

The use of polymers of formula (II) in order to form a slippery coating on at least one part of an intraocular implant injection device is one object of the invention.

Compounds usable for the purposes of the invention are also, for example, those of Examples 1 and 2 of Application EP1521795.

The thermogelling polymer capable of forming a slippery coating according to the invention has a low viscosity in solution in water at a concentration of less than 10% at ambient temperature and is capable of forming physical gels with a high viscosity index at a temperature above 25° C. In particular, said low viscosity may be less than 1000 mPa·s, particularly from 0.1 to 500 mPa·s, for example from 0.1 to 100 mPa·s.

An advantageous aspect of the constituent polymer of the coating according to the invention is its film-forming capacity. Indeed, the slippery property of the coating is also linked to the formation of a smooth and homogeneous film. It was also found that the inter- and intra-chain interactions created during temperature rise (thermogelling phenomenon) contribute very strongly to the formation of a very cohesive film. This physical cross-linking of the polymer makes the film insoluble in water. Thus, the polymer is not carried into the eye when the implant is injected.

The invention also relates to a method for coating the inner surface of one or more parts of an intraocular implant injection device, comprising the following steps:
subjecting the inner surface of the part(s) of the intraocular implant injection device to be coated, in particular the tip and/or the cartridge, to a strongly oxidizing treatment, and
depositing a solution of polymer as defined above.

Preferably, said polymer consists in a plurality of PEO-PPO-PEO triblock polyoxyalkylene type linear chains connected to each other by carbamate and/or urea bonds obtained from reacting the hydroxyl ends of PEO-PPO-PEO triblock polyoxyalkylene with a diisocyanate, and wherein said PEO-PPO-PEO chain complies with formula (I)

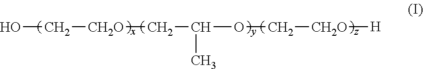

wherein, 10<x<150, 10<y<150, 10<z<150.

Thus, a subsequent aspect of the invention relates, in particular, to a method for depositing the polymer film on the constituent material of the tip and/or cartridge of the ophthalmic injector, which advantageously makes it possible to prevent the coating being detached and carried into the patient's eye.

Preferably, to ensure good adhesion of the coating on the tip and/or the cartridge of the ophthalmic injector, it is possible, for example, to perform a specific treatment of the latter. In a first step, any traces of deposits, such as grease or particles, are removed from the surface of the tip and/or the cartridge by washing, for example by washing with alcohol followed by rinsing with pure water. The surface of the material may then be subjected to a treatment allowing strong oxidation, in order to increase the surface tension of the substrate. Thus, the tip and/or the cartridge may be treated with corona, argon plasma, oxygen, or air. The presence of functions created thanks to oxidation on the surface of the tip and/or the cartridge allows easy creation of hydrogen-type bonds with the coating. Indeed, the systematic presence of urethane bonds of the coating ensures very good adhesion to the substrate treated by corona, or argon plasma, oxygen or air, particularly thanks to hydrogen bonds.

The coating implementing the thermogelling polymer capable of forming a slippery coating according to the invention described above is preferably made via solvent process. Said polymer is solubilized at a concentration ranging from 0.5% to 3% by mass in a solvent of the polymer, and preferably in a mixture consisting of water and ethanol. The tip and/or cartridge is(are) filled with this solution. After a few seconds, for example about 3 seconds, the excess is removed, the polymer deposited on the surface of the tip and/or cartridge is then dried. The film thus deposited is smooth and homogeneous.

The injection tests carried out with these coated tips/cartridges according to the different aspects of the invention allowed easy injection of the implants, including via small diameters (<3 mm) without detachment or solubilization of the coating. No traces of the constituent polymer of the coating could be found by HPLC analysis, either on the implant surface or in the medium wherein the implants were injected.

General and particular aspects of the invention described above apply indifferently to the use of the thermogelling polymers described above in order to form a slippery coating on at least one part of an intraocular implant injection device and to the method for coating the inner surface of at least one part of an intraocular implant injection device implementing them.

### EXAMPLES

### Example 1: Preparation of a Thermogelling Polyurethane

The thermogelling polyurethane is prepared in a reactor equipped with:
an effective stirring system (anchor type and two-stage)
a feed via peristaltic pump for introducing isocyanates
a nitrogen supply
an outlet on a bubbler of a free port for introducing Adekanol® F108 and PEG600.

The reagents used are shown in Table 1.

TABLE 1

| | | Molar | | | |
|---|---|---|---|---|---|
| | Molar | mass | OH Index | | |
| | Ratio | (g/mol) | (mg KOH/g) | Mass (g) | Appearance |
| Adekanol ® F108 | 1 | — | I_{OH} = 6 | 100 + 1% | Pellet |
| | | | to 8* | | |
| Dicyclohexylmethane- | 2.06 | 262 | — | 100 × (I_{OH}/ | liquid |
| 4,4′-diisocyanate | | | | 207.5) ± 1% | |
| polyethylene glycol | 1.73 | 600 | — | 100 × (I_{OH}/ | liquid at |
| monomethyl ether | | | | 107.9) ± 1% | 25° C. |
| (PEG 600) | | | | | |
| Bismuth carboxylate | 175 | | — | 0.0175 ± | liquid |
| | ppm/F108 | | | 0.0005 | |
| Ethanol 99% absolute | 0.1 mL/100 g | | — | 0.1 ml | liquid |
| | F108 | | | | |

*value to be taken from CA for ADEKANOL ® F108 supplied by ADEKA

The synthesis comprises 3 phases:

### A) Drying of Poloxamer and PEG600

The following operations are performed on the day of synthesis.

1) Introduce Adekanol® F108 into the main reactor.

Heat to 120° C. while maintaining the reactor under dynamic vacuum at at least 50 mbar and under continuous stirring. When the material is completely molten, maintain heating and vacuum for another 2 to 4 h (no bubbles in the medium). Maintain vacuum between 20 h and 24 h maximum. A large part of the water contained in the Adekanol® F108 must thus be removed.

2) Place the PEG600 in another reactor allowing heating and application of vacuum at at least 40 mbar. Melt the PEG600 under vacuum at 80° C. and under continuous stirring and maintain the system thus for 2 h from the complete melting of the PEG600.

B) Loading of the Reagents into the Main Reactor

3) Add the catalyst (bismuth carboxylate) under nitrogen flow to the previously dried Adekanol® F108. Stir vigorously.

4) 15 min after adding the catalyst, add dicyclohexylmethane-4,4′-diisocyanate in 15 min.

5) Take a sample 1 h after starting to add isocyanates, if the isocyanate function conversion is greater than or equal to 58%, introduce the PEG600.

When conversion is achieved, add the dry and liquid (80° C.) PEG600 in one go, all under nitrogen flow (synthesis reactor and PEG600 reactor).

6) Maintain under stirring for 2 h.

7) When at least 99% of the isocyanates are used up, add the ethanol to the core of the reaction medium. Stir for 30 min before retrieving the polymer.

8) Check for the absence of isocyanates, and perform 4 vacuum/nitrogen cycles.

### C) Polymer Retrieval

The polymer is hot-cast. The yield from this synthesis is greater than or equal to 97%.

After cooling, it is ground into powder form. This powder can then be solubilized in acetone and reprecipitated in a hydrocarbon such as pentane or heptane.

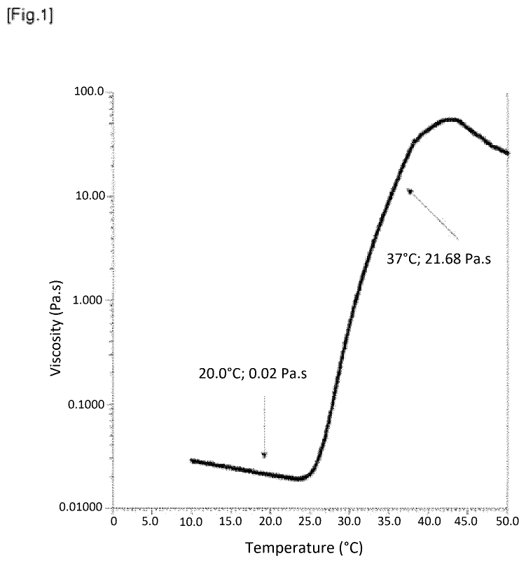

FIG. 1 shows the rheological flow (10 s⁻¹) characterization as a function of temperature, between 10° C. and 50° C., of the polymer of Example 1 in solution in water at a concentration of 7.5%, and confirms its thermogelling property.

### Example 2: Thermogelling Polyurethane

The thermogelling polyurethane is prepared in a reactor equipped with:
an effective stirring system (anchor type and two-stage)
a feed via peristaltic pump for introducing isocyanates
a nitrogen supply
an outlet on a bubbler
a free port for introducing Pluronic® F127 and PEG600.

The reagents used are shown in Table 2.

TABLE 2

| | | Molar | | Number | |
|---|---|---|---|---|---|
| | Molar | mass | Mass | of moles × | Appear- |
| | Ratio | (g/mol) | (g) | 10⁻³ | ance |
| Pluronic ® F127 | 1 | 13,000 | 100 ± | 7.692 | hygro- |
| | | | 1% | | scopic |
| | | | | | powder |
| Dicyclohexyl- | 2.06 | 262 | 4.152 + | 15.846 | liquid |
| methane-4,4′- | | | 1% | | |
| diisocyanate | | | | | |
| polyethylene glycol | 1.84 | 600 | 8.506 ± | 13.976 | Liquid at |
| monomethyl ether | | | 1% | | 25° C. |
| (PEG 600) | | | | | |
| Bismuth | | | 0.047 + | | liquid |
| carboxylate | | | 1% | | |
| Ethanol 99% | | | 0.1 ml | | liquid |
| absolute | | | | | |

The synthesis comprises 3 phases:

### A) Drying of Poloxamer and PEG600

The following operations are performed on the day of synthesis.

1) Introduce the Pluronic® F127 into the main reactor.

Heat to 120° C. while maintaining the reactor under dynamic vacuum at at least 50 mbar and under continuous stirring. When the material is completely molten, maintain heating and vacuum for another 2 to 4 hours (no bubbles in the medium). Maintain vacuum between 20 h and 24 h maximum. A large portion of the water contained in the Pluronic®F127 must thus be removed.

2) Place the PEG600 in another reactor allowing heating and application of vacuum at at least 40 mbar. Melt the PEG600 under vacuum at 80° C. and under continuous stirring and maintain the system thus for 2 hours from the complete melting of the PEG600.

B) Loading of the Reagents into the Main Reactor

3) Add the catalyst (bismuth carboxylate) under nitrogen flow to the previously dried F127. Stir vigorously.

4) 15 minutes after adding the catalyst, add dicyclohexylmethane-4,4′-diisocyanate in 15 min.

5) Take a sample 1 h after starting to add isocyanates, if the isocyanate function conversion is greater than or equal to 58%, introduce the PEG600.

When conversion is achieved, add the dry and liquid (80° C.) PEG600 in one go, all under nitrogen flow (synthesis reactor and PEG600 reactor).

6) Maintain under stirring for 2 h.

7) When at least 99% of the isocyanates are used up, add the ethanol to the core of the reaction medium. Stir for 30 min before retrieving the polymer.

8) Check for the absence of isocyanates, and perform 4 vacuum/nitrogen cycles.

### C) Polymer Retrieval

The polymer is hot-cast. The yield from this synthesis is greater than or equal to 97%.

After cooling, it is ground into powder form. This powder can then be solubilized in acetone and reprecipitated in a hydrocarbon such as pentane or heptane.

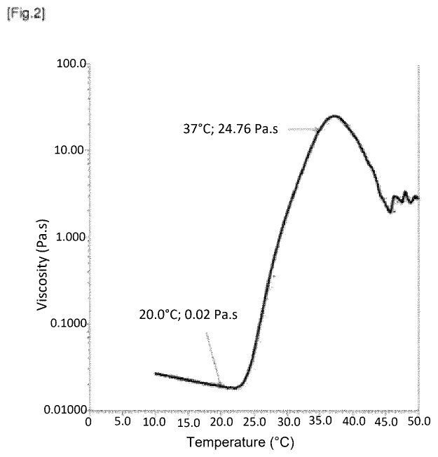

FIG. 2 shows the rheological flow (10 s⁻¹) characterization as a function of temperature, between 10° C. and 50° C., of the polymer of Example 2 in solution in water at a concentration of 7.5%, and confirms its thermogelling property.

### Example 3: Treatment of the Tips and Cartridges Before Application of the Coating According to the Invention

The tips/cartridges are washed twice with ethanol and then rinsed twice with pure water.

They are then dried with compressed air.

The plasma apparatus used is a PlasmaNet® MWGO. The gas used is air at a pressure of 0.4 mbar. The gas use time is 20 s and the plasma treatment lasts 195 s at a power of 3000 W.

The surface tension of the tips/cartridges is determined using testing inks after the plasma treatment to ensure its effectiveness. Indeed, at the end of the cycle, tip/cartridge samples are collected; ACOTEST® 56 mN/m ink is deposited on the surface of the substrates. The plasma treatment is validated if the ink spreads perfectly.

### Example 4: Application of the Coating According to the Invention

The polymer from Example 1 is solubilized at 0.625% in water for at least 48 h at ambient temperature. The solution is then filtered on a 0.8 μm filter. The solution is ready for application.

The tips/cartridges of Accujet® type injectors are filled with the solution prepared above. The excess is removed under vacuum after about 3 s of contact. The deposit is then dried. The film formed is smooth and homogeneous.

### Example 5: Injection Tests

50 hydrophilic implants and 50 hydrophobic implants of diopter 28 were injected using an injector having a tip 2 mm or 2.6 mm in diameter (treated according to the protocol of Example 4).

The injection tests carried out, using an Accujet® type injector, with coatings prepared from different polymers are reported in Table 3 below. These tests are carried out by immersing the tip in a pure water bath at 32° C. to best approximate injection conditions into the human eye.

The measurement was carried out by compression using an Instron 3367 type dynamometer equipped with a force sensor or 0.5 kN sensitivity at a speed of 4 mm/s and at ambient temperature.

TABLE 3

| | Lens | Tip | Coating | Outlet IOL |
|---|---|---|---|---|
| Reference | Type/Diopter | diameter | type | Force (N) |
| DIM22104 A | Hydrophobic*/28 | 2.6 mm | Example 1 | 15 |
| | | | | 15 |
| | | | Mean | 15 |
| | | | Standard | 0 |
| | | | deviation | |
| DIM22104 B | Hydrophobic*/28 | 2.6 mm | Example 2 | 14 |
| | | | | 13 |
| | | | | 14 |
| | | | Mean | 14 |
| | | | Standard | 1 |
| | | | deviation | |
| DIM22103 A | Hydrophilic/28 | 2.0 mm | Example 1 | 11 |
| | | | | 11 |
| | | | Mean | 11 |
| | | | Standard | 0 |
| | | | deviation | |
| DIM22103 B | Hydrophilic/28 | 2.0 mm | Example 2 | 10 |
| | | | | 11 |
| | | | | 10 |
| | | | Mean | 10 |
| | | | Standard | 1 |
| | | | deviation | |

*the lenses chosen are Innoloop ® implants from Innolens

The results show that the deposited coating gives the injector a slippery property making it possible to inject an intraocular implant of at least 6 mm in diameter via a tip 2 mm in diameter for hydrophilic implants and 2.6 mm for hydrophobic implants.

### Example 6: Slippery Coating Resistance

50 hydrophilic implants of diopter 28 were injected using an injector having a tip 2 mm in diameter (treated according to the protocol of Example 4) under the conditions described in Example 5.

The water bath containing the implants was concentrated with a rotary evaporator and then freeze-dried. The residue was analyzed by HPLC (water/acetonitrile). No trace of the coating implementing the polymer of Example 1 was found in the analyzed product.

The results show that the coating according to the invention adheres to the surface of the injector and does not detach during implant injection.

What is claimed is:

1.-20. (canceled)

21. A polymer for use as a slippery coating, said polymer being a water-soluble heat-sensitive polymer comprising at least one heat-sensitive polyoxyalkylene triblock type linear chain consisting of polyethylene oxide (PEO) blocks and polypropylene oxide (PPO) blocks, said chain having the form PEO-PPO-PEO and being extended at at least one of its ends by an organic group via a carbamate bond and/or a urea bond, wherein said polymer is capable of forming a thermoreversible physical gel having a high viscosity index.

22. The polymer according to claim 21, said polymer comprising a plurality of PEO-PPO-PEO triblock polyoxyalkylene type linear chains connected to each other by carbamate and/or urea bonds.

23. The polymer according to claim 22, wherein the chains are connected to each other by carbamate and/or urea bonds obtained from reacting the hydroxyl ends of PEO-PPO-PEO triblock polyoxyalkylene with a diisocyanate, wherein the diisocyanate is chosen from:

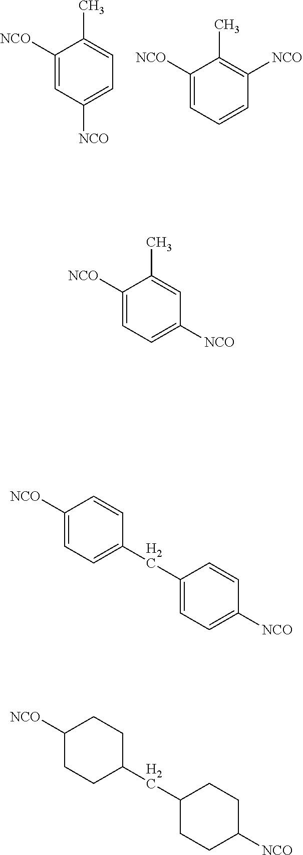

24. The polymer according to claim 23, said polymer chains being either terminated by adding ethanol or extended by adding a polyethylene glycol monomethyl ether.

25. The polymer according to claim 21, wherein said PEO-PPO-PEO chain complies with formula (I) 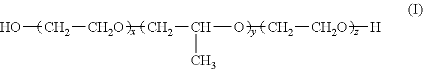
wherein 10<x<150, 10<y<150 and 10<z<150.

26. The polymer according to claim 21, wherein said polymer complies with formula (II) 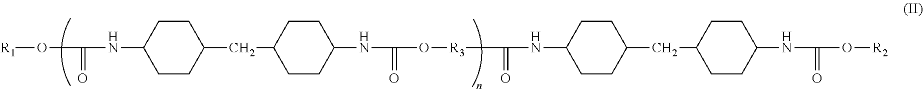
wherein:
R₁ and R₂, identical or different, represent —(CH₂—CH₂—O)ₚ—CH₃ or —CH₂—CH₃;
n is an integer from 2 to 50 and p is an integer from 2 to 70
R₃ represents formula (III): 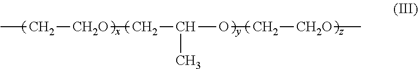
wherein 10<x<150, 10<y<150 and 10<z<150.

27. The polymer according to claim 26, wherein in formula (I) or (III), x=z.

28. The polymer according to claim 21, wherein said polymer has a low viscosity at ambient temperature in solution in water at a concentration of less than 10% and is capable of forming physical gels with a high viscosity index at a temperature greater than 25° C.

29. An intraocular implant injection device comprising a slippery coating comprising the polymer according to claim 21.

30. The intraocular implant device according to claim 29, wherein one or more of the tip and/or the cartridge of an ophthalmic injector is coated with the slippery coating.

31. The intraocular implant injection according to claim 29, wherein an inner surface of which is coated with the slippery coating.

32. The intraocular implant injection device according to claim 29, comprising at least one part, the inner surface of which is coated with the slippery coating.

33. A part of an intraocular implant injection device, the inner surface of which is coated with the polymer according to claim 21.

34. A part of an intraocular implant injection device, the inner surface of which is coated with a polymer according to claim 23.

35. A part of an intraocular implant injection device, the inner surface of which is coated with a polymer according to claim 25.

36. A method for coating an inner surface of one or more parts of an intraocular implant injection device, comprising the following steps:
subjecting the inner surface to be coated to a strongly oxidizing treatment; and
depositing a solution of the polymer as defined in claim 21.

37. The method according to claim 36, wherein the polymer comprises a plurality of PEO-PPO-PEO triblock polyoxyalkylene type linear chains, wherein said PEO-PPO-PEO chain are of formula (I) 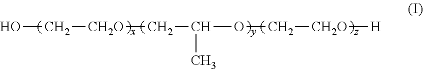
wherein, 10<x<150, 10<y<150, 10<z<150.

38. The method according to claim 36, wherein the strongly oxidizing treatment of the inner surface is a corona, argon plasma, oxygen, or air treatment.

39. The method according to claim 36, wherein the polymer is solubilized in a solvent at a concentration of from 0.5% to 3% by mass to form a polymer solution.

40. The method according to claim 39, wherein the part of the injector to be coated is filled with the polymer solution, the excess removed, then the part dried.
